(19) United States
(12) Patent Application Publication
(10) Pub. No.: US 2012/0210636 A1
(43) Pub. Date: Aug. 23, 2012

(54) Large Scale Syngas BTU Enhancement for Power Generation

(76) Inventor: James Charles Juranitch, Ft. Lauderdale, FL (US)

(21) Appl. No.: 13/384,022

(22) PCT Filed: Jul. 13, 2010
(86) PCT No.: PCT/US10/01956

§ 371 Date: Apr. 30, 2012

Related U.S. Application Data
(60) Provisional application No. 61/270,928, filed on Jul. 14, 2009.
(60) Provisional application No. 61/270,820, filed on Jul. 13, 2009.

(51) Int. Cl. A01G 7/02; C07C 29/152; C07C 1/12; C01B 3/02; C07C 29/151

(52) U.S. Cl. 47/1.4; 252/373; 518/702

(57) ABSTRACT

A method and system for converting low BTU synthesis gas (Syngas), and synthesis gas that has been generated in situ, into a higher BTU product while minimizing the process carbon footprint. Preferably, a plasma gassifier is used to generate the syngas. Sensible heat is recovered and applied to produce electricity. The syngas is water gas shifted to enhance hydrogen production. Gasification is performed in a pyrolysis mode of operation, a nitrogen reduced mode of operation, an oxygen enriched mode of operation, or a coke supplemented mode of operation. The syngas is delivered to a reactor to produce product. The reactor is any of a pellet style reactor, a monolith style reactor, a foam reactor, a ceramic foam reactor, an alumina oxide reactor, and an alpha alumina oxide reactor.

### RELATIONSHIP TO OTHER APPLICATIONS

This application claims the benefit of the filing date of U.S. Provisional Patent Application Ser. No. 61/270,820, filed Jul. 13, 2009, Conf. No. 8494 (Foreign Filing License Granted); U.S. Provisional Patent Application Ser. No. 61/270.928, filed Jul. 14, 2009, Conf. No. 5021 (Foreign Filing License Granted), each in the name of the same inventor as herein. The disclosures in the identified U.S. Provisional Patent Applications are incorporated herein by reference.

### BACKGROUND OF THE INVENTION

1. Field of the Invention

This invention relates generally to power generation systems, and more particularly, to a method of increasing the BTU content of Syngas, and assuring its consistent component quality, from a syngas generating system.

2. Description of the Related Art

In the current energy environment there is ever more desire to use renewable, or carbon neutral energy sources. In the process of using these energy sources many times Synthesis Gas or syngas is produced as a way of transferring chemical energy. For many reasons syngas to date has had a difficult time making its way into production of large scale energy. One of the primary reasons is its energy density. It typically has a heating value from approximately 150 BTU/ft³ to 400 BTU/ft³. When compared to natural gas, or methane at approximately 1000 BTU/ ft³ the syngas is typically ⅙ to ⅓ the energy density. It also has varying BTU content and composition in most applications that generate syngas.

These problems for the most part have relegated syngas to only small scale electrical energy production. In most cases production is below 10 MW. In these small scale electrical energy systems typically one or more internal combustion engines are used to drive electric generators. These systems are somewhat tolerant of low and varying fuel BTU content combined with varying compositions that effect combustion. Even with these positive traits the internal combustion engines must be approximately 3 to 6 times the size, quantity, and cost of a similar generator sets that would be optimized for natural gas, or methane. As is obvious the varying fuel content and BTU level of syngas is also a tremendous reliability and operational problem, independent of the power density.

When turbines are used the same problems are only magnified. The is unfortunate because a modern combined cycle turbine electric generation system is typically one of the most energy efficient methods of producing electricity from a liquid, or gaseous fuel source known today.

The present invention teaches a way of solving all of the above problems in an energy efficient, and cost effective way. It is well suited to large scale integration. It also produces a minimal to carbon neutral footprint.

Two scientists, Drs. Circeo and Camacho, have distinguished themselves as pioneers in the field of employing plasma for energy reclamation purposes, and more specifically, in the use of plasma in a unique application referred to as “in situ.” The concept of these two scientists is described in their U.S. Pat. No. 4,067,390. However, this concept has not enjoyed widespread industrial use for a number of reasons. First, with respect to in situ plasma applications, relatively poor energy density is achieved. Also, the chemical composition of the syngas that is produced from the known in situ application varies. Since the in situ sites are usually remote the energy density problem is accentuated by associated energy transportation issues.

As a result of the foregoing, syngas has not widely been applied to the production of large scale energy, or chemical feedstock use, particularly because its energy density is low. Typically, syngas has a heating value of approximately between 150 BTU/ft³ to 400 BTU/ft³. When compared to natural gas, or methane at approximately 1000 BTU/ft³ the syngas is typically ⅙ to ⅓ the energy density. It also has varying BTU content and composition in most applications that generate syngas.

These problems for the most part have relegated syngas to only small scale electrical energy production. In most cases production is below 10 MW. In these small scale electrical energy systems typically one or more internal combustion engines are used to drive electric generators. These systems are tolerant of low and varying fuel BTU content combined with varying compositions that effect combustion. Even with these positive traits the internal combustion engines must be approximately 3 to 6 times the size, quantity, and cost of a similar generator sets that would be optimized for natural gas or methane. It is also evident that the varying fuel content and BTU level of syngas creates a significant reliability and operational problems, independent of the low power density.

When turbines are used, the foregoing problems are accentuated. This is unfortunate because a modern combined cycle turbine electric generation system is among the most energy efficient methods of producing electricity from a liquid or gaseous fuel source.

If the syngas is produced to be used for a feedstock in a chemical process the same issues are also detrimental to its success. When compared to the classic feedstock of natural gas for the chemical industry the parallel of issues is obvious.

This invention teaches a method of solving all of the above problems in an energy efficient, and cost effective way. It is well suited to large scale integration. It also produces a minimal carbon footprint, or is neutral in that regard.

### SUMMARY OF THE INVENTION

The foregoing and other objects are achieved by this invention which provides a method that includes the steps of:

producing syngas in a syngas generating system that employs a gassifier;

recovering excess heat from the syngas using a heat recovery arrangement; and

subjecting at least a portion of the syngas to a reaction in a reactor.

In an advantageous embodiment of the invention, the reactor is a selectable one of a Fisher Tropsh style reactor, a Richardson reactor, a Sabatier reactor. In other embodiments, the reactor produces fuels, and is a selectable one of a methane reactor arrangement, an ethane reactor arrangement, a propane reactor arrangement, a butane reactor arrangement, a cetane reactor arrangement, and a methanol reactor arrangement.

In a highly advantageous embodiment of the invention, the gassifier is a plasma gassifier. Also, the heat recovery arrangement is a sensible heat recovery arrangement that issues excess heat as steam. The excess heat is applied to make electricity.

In a further embodiment, the step of recovering excess heat from the syngas comprises the step of recovering low level sensible heat from the syngas. The excess heat is applied to make electricity.

The syngas is subjected to the further step, in some embodiments, of being cleaned.

In other embodiments, there is provided the further step of water gas shifting the syngas to enhance hydrogen production.

In an advantageous embodiment of the invention, there is provided the further step of operating the gassifier in a selectable one of a pyrolysis mode of operation, a nitrogen reduced mode of operation, an oxygen enriched mode of operation, and a coke supplemented mode of operation.

A product is produced in accordance with the invention by the further step of conducting the syngas to a reactor to produce a product. The reactor is a selectable one of a pellet style reactor, a monolith style reactor, a foam reactor, a ceramic foam reactor, an alumina oxide reactor, and an alpha alumina oxide reactor. Moreover, the reactor is configured in respective embodiments of the invention to be a selectable one of a Sabatier reactor, a Fisher Tropsh reactor, a Methanol reactor, and a Richardson Reactor. Other steps that are applied in the practice of this method aspect of the invention include:

water gas shifting the syngas to enhance hydrogen production; and

conducting a product CO₂ from said step of water gas shifting to a selectable one of an algae bioreactor and a pond.

In the practice of the invention, there is provided the further step of enhancing a concentration of H₂ by using a selectable one of an aqueous solution, a PSA, and a membrane separation system. In such embodiments, the reactor is configured to be a Methanol reactor, and there is provided the further step of condensing and separating a gaseous methanol from the balance of the syngas product. Subsequently, a reactor product or fuel is conducted into an energy converting system. The energy converting system is, in respective embodiments of the invention, a selectable one of an internal combustion engine generator and a combined cycle electricity generating system.

In accordance with a further method aspect of the invention, there is provided a method of increasing the BTU content and quality of Syngas. This further method aspect includes the steps of:

producing syngas in an in situ plasma gassifier operated in a pyrolysis mode; and

recovering heat from the syngas using a heat recovery arrangement.

In one embodiment, there is provided the further step of subjecting at least a portion of the syngas to a reaction in a reactor. This includes, in some embodiment, the further step of conducting the syngas to a reactor to produce product. In such embodiments, the reactor is a selectable one of a pellet style reactor, a monolith style reactor, a foam reactor, a ceramic foam reactor, an alumina oxide reactor, and an alpha alumina oxide reactor.

In embodiments of the invention where the reactor is a Methanol reactor, there is provided the further step of condensing and separating a gaseous methanol from the balance of the syngas product. There are additionally provided the steps of:

separating CO; and

reprocessing the separated CO through a water gas shift reactor.

In one embodiment of this further method aspect of the invention, H₂ is used to make a final product. The final product can, in some embodiment, by methanol.

In another embodiment of the invention, there is provided the further step of operating the gassifier in a selectable one of a pyrolysis mode of operation, a nitrogen reduced mode of operation, an oxygen enriched mode of operation, and a coke supplemented mode of operation.

In other embodiments, there is provided the further step of conducting the syngas to a reactor to produce a product. The reactor is a selectable one of a pellet style reactor, a monolith style reactor, a foam reactor, a ceramic foam reactor, an alumina oxide reactor, and an alpha alumina oxide reactor. In still further embodiments, the reactor is configured to be a selectable one of a Sabatier reactor, a Fisher Tropsh reactor, a Methanol reactor, and a Richardson Reactor. There are additionally provided in some embodiments the further steps of:

water gas shifting the syngas to enhance hydrogen production; and

conducting a product CO₂ from said step of water gas shifting to a selectable one of an algae bioreactor and a pond.

In some embodiments, there is provided the further step of enhancing a concentration of H₂ by using a selectable one of an aqueous solution, a PSA, and a membrane separation system.

In some embodiments the reactor is configured to be a Methanol reactor, and there is provided the further step of condensing and separating a gaseous methanol from the balance of the syngas product.

The reactor product or fuel is conducted into an energy converting system, the energy converting system being a selectable one of an internal combustion engine generator and a combined cycle electricity generating system.

### BRIEF DESCRIPTION OF THE DRAWING

Comprehension of the invention is facilitated by reading the following detailed description, in conjunction with the annexed drawing, in which:

FIG. 1 is a simplified schematic representation of a syngas BTU enhancement system constructed in accordance with the invention; and

FIG. 2 is a simplified schematic representation of an in situ syngas generation system in which the syngas BTU content is enhanced in accordance with the invention.

### DETAILED DESCRIPTION

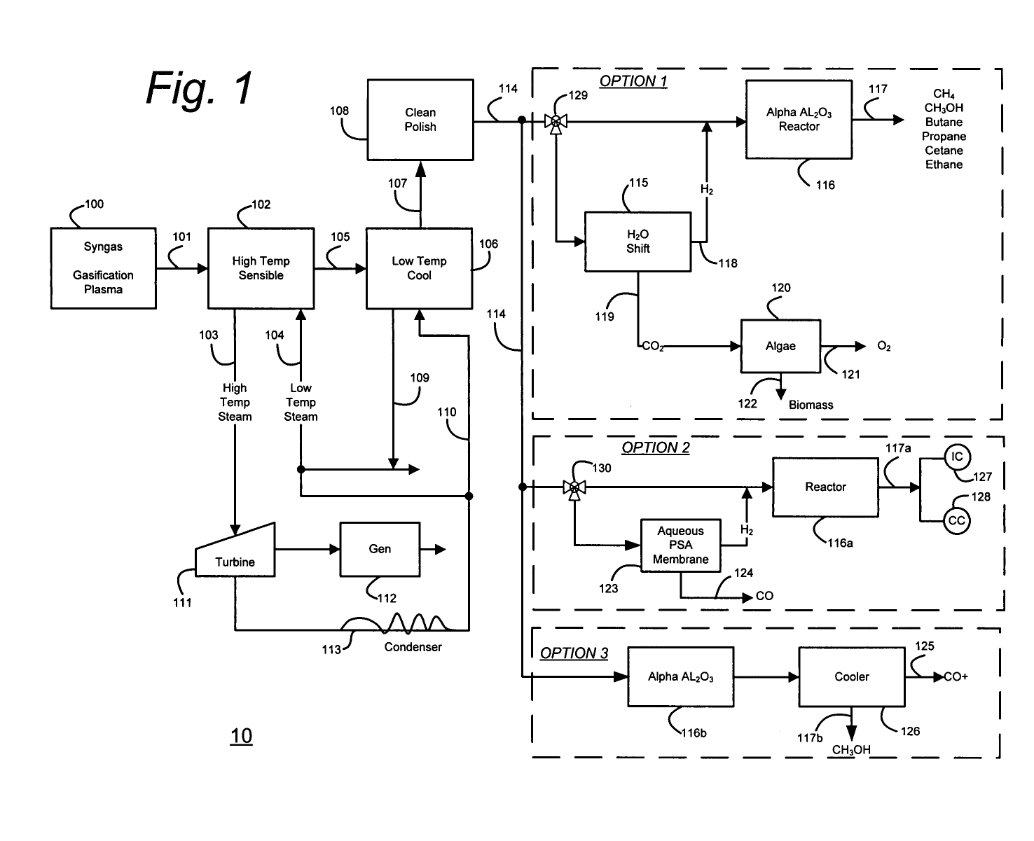

FIG. 1 is a simplified schematic representation of a syngas BTU enhancement system 10 constructed in accordance with the invention. As shown in this figure, syngas is produced at a plasma gassifier 100. In the practice of the invention, gassifier 100 is a conventional gasification system, and in a preferred embodiment of the invention, it is a plasma reactor. The feedstock (not shown) for the syngas is, in some embodiments, a fossil fuel such as coal, or a renewable source of energy such as algae, biomass, or Municipal Solid Waste (MSW).

Although not specifically shown or designated in the figure, the syngas in various embodiments of the invention can be produced by an oxygen deprived system (pyrolysis), an oxygen enriched system, a nitrogen reduced environment, a coke enhanced system, or any other desired gasification process.

The syngas available at syngas outlet 101 is, in this embodiment, delivered to a sensible heat recovery system 102. This heat recovery system is optional, but beneficially serves to make the process energy positive, or at least energy neutral, depending on the gasification method that is implemented. Sensible heat at heat outlet 103 is routed in the form of steam, in this embodiment, to turbine 111 that is in mechanical communication with electrical generator 112. A low temperature heat recovery system 106 also is optional, and its use in the practice of the invention will depend greatly on the gasification process and feedstock (not shown) that is used.

The syngas at syngas outlet 107 is then conducted to a cleaning stage 108, which in this embodiment is a cleaning and polishing module. In respective embodiments of the invention, at least three options are available:

In a first option, syngas in conduit 114 is, in this embodiment, divided in a flow control valve 129. Part of the flow is delivered to a water gas shift system 115 to produce additional H₂ at outlet 118. The resulting CO₂ is, in this embodiment, delivered to an algae bioreactor 120, which may be a pond, where is converted to O₂ at an outlet 121, and to biomass at a further outlet 122. The resulting H₂ boosted syngas then enters a reactor 116, which in respective embodiments of the invention is a pellet, monolith, foam, ceramic foam, alumina oxide foam, or an alpha alumina oxide foam reactor. In the practice of the invention, reactor 116 is any of a Fisher Tropsh style reactor, a Richardson reactor, a Sabatier reactor, or many other styles of reactor arrangements to produce fuels such as methane, ethane, propane, butane, cetane, methanol, and others.

In addition to the foregoing, there is provided in accordance with the invention a second option wherein syngas in conduit 114 is, in some embodiments, divided through flow control valve 130 into a Pressure Swing Absorption (PSA) system 123, which in various embodiments of the invention can be configured as a membrane system, an aqueous solution system, or any other conventional form of H₂ separation system. The separated H₂ is then conducted to reactor 116a. The fuel produced at outlet 117a of reactor 116a is then delivered to electrical power generator 127, which in this embodiment is an internal combustion power system, or to a combined cycle power generator 128. It is to be understood that the consistent fuel at outlet 117a is not limited to the applications herein mentioned, and can be used for many conventional power conversion systems such as steam boilers, etc.

As a third option, the syngas in conduit 114 is conducted to a reactor 116b that in this embodiment of the invention is configured for the production of methanol. The methanol thereby produced is then conducted to a cooler 126 that condenses out liquid methanol at a methanol outlet 117b and expels the balance of the un-reacted CO and syngas byproducts at an outlet 125. CO product 124 (Option 2) and 125 (Option 3) can be used as a low BTU fuel, or it can be sold for industrial uses. The CO is, in some embodiments, water gas shifted and reprocessed with the additional H₂ produced through reactor 116 for increased methanol production as seen in sub-loop and reactor 115 which then processes the CO₂ in algae bioreactor 120.

FIG. 2 is a simplified schematic representation of an in situ syngas generation system 20 in which the syngas BTU content is enhanced in accordance with the invention. Elements of structure that have previously been discussed are similarly designated. As shown in this figure, syngas is produced by an in situ plasma syngas generator 100. An illustrative known suitable syngas generator is described in U.S. Pat. No. 4,067,390. However, the present invention is not limited to the in situ system described in that patent. Many new concepts such as tent syngas collection systems, and electronic optical feedback systems will undoubtedly enhance in situ productivity. These improvements are also able to benefit from this invention. Unfortunately no matter how efficiently the in situ syngas is recovered with ever better technical approaches, it still has all the fundamental problems described above once it is recovered. The present invention provides a solution to those problems.

The syngas produced could be from an oxygen deprived system (pyrolysis), an oxygen enriched system, a nitrogen reduced environment, a coke enhanced system, or any other desired gasification process. Syngas is available at outlet 101 of syngas generator 100 and is then, in this embodiment of the invention, supplied to a sensible heat recovery system 102. Sensible heat recovery system 102 is not required, but will serve to render the process herein described to be energy positive, or at least energy neutral, depending on the gasification method that is implemented. The sensible heat at sensible heat outlet 103 can, in some embodiments of the invention, be used for power generation or process work, illustratively as described above in relation to FIG. 1. The quantity of heat recovered will depend greatly on the gasification process, the energy content of the feedstock, and the depth of the shaft (not shown) from which the energy is recovered. In any case the syngas must be cooled before it is supplied to the next stage.

As shown in FIG. 2, cooled syngas 105 is then supplied to a cleaning and polishing module 108. Cleaned syngas 114 is then provided to at least three system options, as described above.

Pursuant to a first option, syngas 114 is divided in a flow control valve 129. Part of the flow is supplied to water gas shift system 115 to produce additional H₂. The resulting CO₂ 119 is then supplied to an algae bioreactor 120, which in some embodiments of the invention is a pond, to be converted to O₂ 121 and biomass 122. H₂ boosted syngas 118 then enters reactor 116. In respective embodiments of the invention, reactor 116 is any of a pellet reactor, a monolith reactor, a foam reactor, a ceramic foam reactor, an alumina oxide foam reactor, and an alpha alumina oxide foam reactor. In other embodiments of the invention, reactor 116 is set up as a Fisher Tropsh style reactor, a Richardson reactor, a Sabatier reactor, or any of several other styles of reactor arrangements that produce fuels, such as methane, ethane, propane, butane, cetane, methanol, and others.

Pursuant to a second option, syngas 114 is divided through flow control valve 130, and a portion thereof is supplied to a Pressure Swing Absorption (PSA) system 123. In various embodiments of the invention, PSA system 123, is any of a membrane system, an aqueous solution system, and any other conventional form of H2 separation arrangement. The separated H₂ is then supplied to a reactor 116a, which in some embodiments of the invention, is reactor 116a. A fuel 117a that is produced by reactor 116 (Option 1) or reactor 116a (Option 2) is supplied to electrical power generator 127, which is an internal combustion power system, or to combined cycle power generator 128. It is to be understood that the use of fuel 117a is not limited to these applications, and is useful for many conventional power conversion systems (not shown) such as steam boilers, or piped, or trucked off-site in any form such as methanol, or natural gas, to be used in any other industrial, or energy application. A particular advantage of this invention is that fuel 117a is at this stage characterized by high energy density, and is an easily transported consistent fuel.

In some embodiments of the invention, there is available a third option wherein reactor 116b, which can also be reactor 116, is used in combination with a cooler 126 to produce liquid methanol, as herein described. The transportation of this methanol energy source is as simple as transporting gasoline or diesel fuel.

As noted above, syngas 114 is in some embodiments of the invention supplied to reactor 116b (or reactor 116), which is configured for the production of methanol. The methanol is then delivered to cooler 126, which condenses out liquid methanol 117b and expels the balance of the un-reacted CO and syngas byproducts at an outlet 125. CO product 124 (from PSA system 123) and CO+ product 125 are useful as a low BTU fuel, and can be sold for industrial uses. In some embodiments, the CO is water gas shifted and reprocessed with the additional H₂ produced through reactor 116b for increased methanol production as seen in the sub loop of water gas shift system 115, which then supplies the CO₂ to bioreactor 120 for processing.

Although the invention has been described in terms of specific embodiments and applications, persons skilled in the art may, in light of this teaching, generate additional embodiments without exceeding the scope or departing from the spirit of the invention claimed herein. Accordingly, it is to be understood that the drawing and description in this disclosure are proffered to facilitate comprehension of the invention, and should not be construed to limit the scope thereof.

What is claimed is:

1. A method of producing high BTU content syngas, the method comprising the steps of:
producing syngas in a syngas generating system that employs a gassifier;
recovering excess heat from the syngas using a heat recovery arrangement; and
subjecting at least a portion of the syngas to a reaction in a reactor.

2. The method of claim 1, wherein the reactor is a selectable one of a Fisher Tropsh style reactor, a Richardson reactor, and a Sabatier reactor.

3. The method of claim 1, wherein the reactor produces fuels, and is a selectable one of a methane reactor arrangement, an ethane reactor arrangement, a propane reactor arrangement, a butane reactor arrangement, a cetane reactor arrangement, and a methanol reactor arrangement.

4. The method of claim 1, wherein the gassifier is a plasma gassifier.

5. The method of claim 1, wherein the heat recovery arrangement is a sensible heat recovery arrangement that issues excess heat as steam.

6. The method of claim 5, wherein the excess heat is applied to make electricity.

7. The method of claim 5, wherein said step recovering excess heat from the syngas comprises the step of recovering low level sensible heat from the syngas.

8. The method of claim 7 wherein the excess heat is applied to make electricity.

9. The method of claim 1, wherein there is provided the further step of cleaning the syngas.

10. The method of claim 1, wherein there is provided the further step of water gas shifting the syngas to enhance hydrogen production.

11. The method of claim 1, wherein there is provided the further step of operating the gassifier in a selectable one of a pyrolysis mode of operation, a nitrogen reduced mode of operation, an oxygen enriched mode of operation, and a coke supplemented mode of operation.

12. The method of claim 1, wherein there is provided the further step of conducting the syngas to a reactor to produce a product.

13. The method of claim 12, wherein the reactor is a selectable one of a pellet style reactor, a monolith style reactor, a foam reactor, a ceramic foam reactor, an alumina oxide reactor, and an alpha alumina oxide reactor.

14. The method of claim 13, wherein the reactor is configured to be a selectable one of a Sabatier reactor, a Fisher Tropsh reactor, a Methanol reactor, and a Richardson Reactor.

15. The method of claim 14, wherein there are provided the further steps of:
water gas shifting the syngas to enhance hydrogen production; and
conducting a product CO₂ from said step of water gas shifting to a selectable one of an algae bioreactor and a pond.

16. The method of claim 14, wherein there is provided the further step of enhancing a concentration of H₂ by using a selectable one of an aqueous solution, a PSA, and a membrane separation system.

17. The method of claim 14, wherein the reactor is configured to be a Methanol reactor, and there is provided the further step of condensing and separating a gaseous methanol from the balance of the syngas product.

18. The method of claim 14, wherein a reactor product or fuel is conducted into an energy converting system.

19. The method of claim 18, wherein the energy converting system is a selectable one of an internal combustion engine generator and a combined cycle electricity generating system.

20. A method of increasing the BTU content and quality of Syngas, the method comprising the steps of:
producing syngas in an in situ plasma gassifier operated in a pyrolysis mode; and
recovering heat from the syngas using a heat recovery arrangement.

21. The method of claim 20, wherein there is provided the further step of subjecting at least a portion of the syngas to a reaction in a reactor.

22. The method of claim 20, wherein there is provided the further step of conducting the syngas to a reactor to produce product.

23. The method of claim 22, wherein the reactor is a selectable one of a pellet style reactor, a monolith style reactor, a foam reactor, a ceramic foam reactor, an alumina oxide reactor, and an alpha alumina oxide reactor.

24. The method of claim 23, wherein the reactor is a Methanol reactor.

25. The method of claim 24, wherein there is provided the further step of condensing and separating a gaseous methanol from the balance of the syngas product.

26. The method of claim 24, wherein there are provided the further steps of:
separating CO; and
reprocessing the separated CO through a water gas shift reactor.

27. The method of claim 26, wherein H₂ is used to make a final product.

28. The method of claim 27, wherein the final product is methanol.

29. The method of claim 20, wherein there is provided the further step of operating the gassifier in a selectable one of a pyrolysis mode of operation, a nitrogen reduced mode of operation, an oxygen enriched mode of operation, and a coke supplemented mode of operation.

30. The method of claim 20, wherein there is provided the further step of conducting the syngas to a reactor to produce a product.

31. The method of claim 30, wherein the reactor is a selectable one of a pellet style reactor, a monolith style reactor, a foam reactor, a ceramic foam reactor, an alumina oxide reactor, and an alpha alumina oxide reactor.

32. The method of claim 31, wherein the reactor is configured to be a selectable one of a Sabatier reactor, a Fisher Tropsh reactor, a Methanol reactor, and a Richardson Reactor.

33. The method of claim 32, wherein there are provided the further steps of:
water gas shifting the syngas to enhance hydrogen production; and
conducting a product CO₂ from said step of water gas shifting to a selectable one of an algae bioreactor and a pond.

34. The method of claim 32, wherein there is provided the further step of enhancing a concentration of H₂ by using a selectable one of an aqueous solution, a PSA, and a membrane separation system.

35. The method of claim 32, wherein the reactor is configured to be a Methanol reactor, and there is provided the further step of condensing and separating a gaseous methanol from the balance of the syngas product.

36. The method of claim 32, wherein a reactor product or fuel is conducted into an energy converting system.

37. The method of claim 36, wherein the energy converting system is a selectable one of an internal combustion engine generator and a combined cycle electricity generating system.
